(19) United States
(12) Patent Application Publication
(10) Pub. No.: US 2005/0255471 A1
(43) Pub. Date: Nov. 17, 2005

(54) Method for analysis of transcription variations in a set of genes

(76) Inventor: Michel Bellis, Montpellier (FR)

Correspondence Address: Edward J Howard;Plevy Howard & Darcy, Post Office Box226, Fort Washington, PA, 19034, US

(21) Appl. No.: 10/516,278

(22) PCT Filed: Jun. 2, 2003
(86) PCT No.: PCT/FR03/01655

§ 371 Date: May 9, 2005

(30) Foreign Application Priority Data
May 31, 2002 (FR) 0206749

(51) Int. Cl. C12Q 1/68; G06F 19/00; G01N 33/48; G01N 33/50

(52) U.S. Cl. 435/6; 702/20

(57) ABSTRACT

The invention relates to a method for analysing the variations in concentration of RNA messengers obtained by transcription of a set of genes comprising the following steps:—measure the concentration of RNA messengers for each of the genes in the so-called reference cells and in test cells and report the results in a reference list and a test list, calculate a variation value for each gene which is a measure of the difference in concentration of m-RNA for said gene between the reference list and the test list, calculate a normalised variation value for each gene such that the cumulative frequency distribution of a sub-set of normalised variation values corresponding to genes has similar or identical m-RNA concentrations whatever the sub-set under consideration and identification of the genes with m-RNA concentration variations significantly different to normalised variation values.

The present invention relates to the analysis of the variations of mRNA concentrations of a set of genes performed by means of DNA chips.

The analysis bears on any type of living cells, such as a bacteria, a yeast, or a cell of a portion of a human body. One or several DNA molecules are present in each cell. Each DNA cell is formed of two complementary polynucleotide strands, an “antisense” strand (−) and a “sense” strand (+). Each polynucleotide strand is formed of a polymeric chain of nucleotides. Each nucleotide is formed of a phosphate, of a sugar (deoxyribose), and of a base, the bases being possibly a guanine (G), an adenine (A), a cytosine (C), and a thymin (T). The two strands of the DNA molecule pair via hydrogen bonds between complementary bases, a guanine being able to pair with a cytosine (G≡C) and an adenine being able to pair with a thymine (A=T).

When a cell is active and lives, each gene synthesizes messenger RNA or mRNA molecules, which are copies, base for base, of the sense strand (+) of the gene. This phenomenon is called the gene transcription or expression. More exactly, the transcription of a gene is only performed for certain groups of consecutive bases, or sequences, of the strand of the expressing gene, the sense strand (+). The mRNA generated by a gene is in fact a regrouping of sequence copies. According to cells, the genes do not all express in the same proportions. Thus, the mRNA concentration relative to a given gene may be zero, or vary between 1 and 10,000 per cell.

A known method to measure the mRNA concentration consists of using DNA chips. Cells are sampled from a culture or from a human body by biopsy. The transcription activity of these cells is then stopped, for example, by freezing. A sample containing in solution the mRNAs extracted from a number of cells is then prepared.

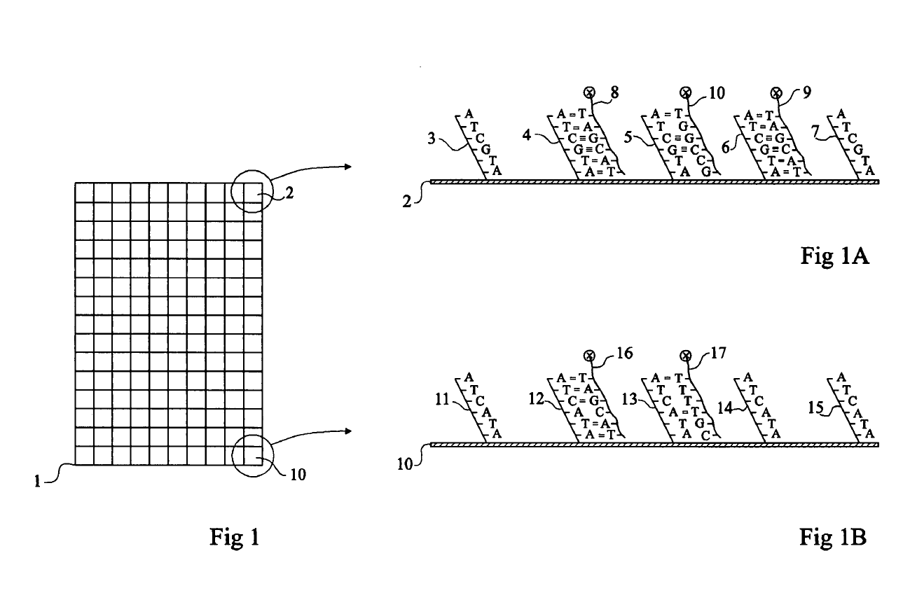

A DNA chip, an example of which is illustrated in FIG. 1, is further prepared to analyze a set of genes. On each chip, each gene is analyzed by means of two sets of some twenty hybridization units. A hybridization unit regroups a set of identical DNA strands called probes. These DNA strands are the complementary strands of a gene sequence which is found in the mRNAs of the analyzed cells. These DNA strands have sequences identical to those of the antisense strand (−) of the gene. A first set of so-called perfect hybridization units (UP), contains probes which correspond to different sequences of a gene. A second set of so-called imperfect hybridization units (UI) contains probes which differ from the probes of the first set for at least one of the bases, each perfect hybridization unit being associated with an imperfect hybridization unit. In the example of FIG. 1, a perfect hybridization unit 2, shown in FIG. 1A, contains probes 3, 4, 5, 6, and 7. Perfect hybridization unit 2 is associated with an imperfect hybridization unit 10, shown in FIG. 1B, which contains probes 11, 12, 13, 14, and 15 which differ by a base (A, G) from probes 3 to 7.

The messenger RNAs of the previously-prepared sample are “marked”, for example, made fluorescent. The strand fluorescence is represented by a cross in a circle placed by the fluorescent strand. The marked messenger RNAs are called targets.

The DNA chip is then placed in the target sample in conditions favoring the hybridization between complementary DNA strands. Thus, a total hybridization of targets 8 and 9 with two probes, respectively 4 and 6, attached on perfect hybridization unit 2 can be seen in FIG. 1. A partial hybridization may occur between a target 10 and a probe 5 which are not totally complementary. A target 16 which is a messenger RNA perfectly complementary to one of the sequences of a gene represented by probes 3 and 7 of perfect hybridization unit 2 may partially hybridize with a probe 12 of imperfect hybridization unit 10. Similarly, another target 17 may partially hybridize with a probe 13 of imperfect hybridization unit 10. A washing step may enable separating the strands which are poorly complementary and thus limit the number of miscouplings.

A photograph of each of the hybridization units of the DNA chip is then taken to determine a fluorescence intensity for each hybridization unit. After measurement of the fluorescence intensities, two fluorescence intensity values i_{UP} and i_{UI} are obtained for each pair of perfect and imperfect hybridization units corresponding to a gene sequence. For each gene sequence, a fluorescence intensity equal to the difference between fluorescence intensity values i_{UP} and i_{UI} is calculated. This method for measuring the fluorescence intensity of each sequence enables obtaining a better signal-to-noise ratio. A fluorescence intensity value is then calculated for each gene by taking the average of the fluorescence intensities of each of the sequences of this gene. A list providing a fluorescence intensity value for each of the genes is thus obtained. The fluorescence intensity being proportional to the concentration of mRNAs provided by the gene transcription, a list providing the mRNA concentration for each gene may easily be obtained. In the case where a gene expresses very little, the fluorescence intensity of the imperfect hybridization units may be greater than that of the perfect hybridization units. The average fluorescence intensity of such a gene may be negative. In this case, it is generally considered that the gene does not express, and thus that the associated mRNA concentration is zero.

Currently, the variations of the mRNA concentrations are desired to be analyzed between so-called reference cells and so-called test cells. This variation analysis will be the object of what follows of the present description and of the invention. The reference cells may for example be healthy liver cells while the test cells are ill liver cells. The same DNA chip models are used, and the previously-described sequence of operations is performed in both cases. The study of the mRNA concentration variations for each gene enables identifying which are the genes for which the mRNA concentration has changed, due to a modification in the transcription activity, or to a change in the mRNA lifetime. The mRNA lifetime fluctuates, among others, according to a more or less significant protein synthesis activity.

Conventionally, the analysis of the mRNA concentration variations for each of the genes is performed by calculating the ratio of the mRNA concentrations of a same gene. This method is known as the “fold change” method. The mRNA concentration variation is considered as being significant when the ratio of the mRNA concentrations is greater than a predetermined threshold. This threshold is identical for all the genes and this method thus does not enable taking into account the specificity of each of them.

The mRNA creation and destruction processes are randomly interrupted at the cell sampling and the mRNA concentration may slightly fluctuate from one cell to another. In the case where a gene generates in average 10 mRNAs in each cell, a difference of a single mRNA between two cells results in a 1.1 ratio, that is, a 10% difference, and the involved gene will be considered as exhibiting a significant mRNA concentration. Conversely, for a gene having in average 1,000 mRNAs per cell, a difference of 10 mRNAs results in a 1.01 ratio, that is, a 1% difference, and this will pass unnoticed while it may be quite abnormal.

The “fold-change” type analysis is thus little reliable since genes exhibiting a significant variation in their concentration may be unidentified.

Further, the mRNA concentration relative to a gene may vary naturally within proportions which are specific thereto. With a simple analysis of fold change type, it is impossible to know to what extent the mRNA concentration variation relative to a gene remains or not within acceptable proportions.

A way to know the natural variation range of the mRNA concentration relative to a gene, or more specifically the cumulative frequency distribution, would be to perform a large number of measurements of mRNA concentrations, for each gene from identical reference cells. In the case where 100 measurements have been performed for each gene, threshold values corresponding to probabilities per increment of 0.01 may be defined so that a same gene associated with identical cells has an mRNA concentration greater than these threshold values. In a measurement of the mRNA concentration of different cells, which probability there is to obtain an mRNA concentration greater than the selected threshold value without for all this for this mRNA concentration to be abnormal can be known.

In practice, it is impossible to perform so many measurements and the selected threshold value is little reliable.

An object of the present invention is to provide a method for analyzing the variations of mRNA concentrations relative to a set of genes which enables taking into account the specificity of gene.

Another object of the present invention is to provide such a method which enables identifying genes exhibiting a significant variation in their mRNA concentrations with a reduced number of measurements.

Another object of the present invention is to provide such a method which enables very accurately defining a threshold value.

To achieve these objects, the present invention provides a method for analyzing the variations of concentrations of messenger RNks obtained by transcription of a set of genes, comprising the steps of:
a) measuring the messenger RNA concentration for each of the genes in so-called reference cells and writing the results in a reference list (L_{ref});
b) measuring the messenger RNA concentration for each of the genes in so-called test cells and writing the results in a test list (Lₜₑₛₜ);
c) calculating for each gene a variation value (Varₖ), k being an integer ranging between 1 and n, which is a measurement of the difference between the mRNA concentrations of said gene between the reference list (L_{ref}) and the test list (Lₜₑₛₜ);
d) classifying the genes in first and second groups, according to whether the genes have variation values respectively corresponding to an increase or to a decrease in their mRNA concentrations between the reference list and the test list;
e) calculating for each gene of the second group a new variation value (Varₖ) which is a measurement of the difference between the mRNA concentrations of said gene between the test list and the reference list;
f) calculating for each gene a normalized variation value (Zₖ) such that the cumulative frequency distribution of a subset of normalized variation values corresponding to genes having close mRNA concentrations is identical whatever the considered subset;
g) identifying the genes exhibiting significant mRNA concentration variations based on the normalized variation values.

According to an embodiment of the method of the present invention, the step of identifying the genes consists of selecting the genes having a normalized variation value greater than a determined threshold value (Zₛₑᵤᵢₗ).

According to an embodiment of the method of the present invention, the determination of the threshold value (Zₛₑᵤᵢₗ) comprises the steps of:
h) measuring the mRNA concentration for each of the genes of two identical so-called calibration cell groups and writing the respective results in a first (L_{étal,1}) and second (L_{étal,1}) sampling lists;
i) calculating for each gene a variation value (Var_{étal,k}) according to the method of steps c) to e) based on the first (L_{étal,1}) and second (L_{étal,2}) sampling lists;
j) calculating for each gene a normalized calibration variation value (Z_{ref,k}) according to the method of step f);
k) constructing the so-called calibration cumulative frequency distribution of the normalized calibration variation values associating with each normalized calibration variation value (Z_{ref,k}) a so-called selection error probability (p_{seuil,k}) for normalized calibration variation values greater than the considered normalized variation value to exist;
l) selecting the desired selection error probability (pₛₑᵤᵢₗ); and
m) defining the threshold value (Zₛₑᵤᵢₗ) corresponding to the desired selection error probability (pₛₑᵤᵢₗ) by means of the cumulative calibration frequency distribution.

According to an embodiment of the method of the present invention, the step of selecting the selection error probability (pₛₑᵤᵢₗ) comprises the steps of:
defining the maximum false positive rate acceptable for the gene identification; and
identifying the maximum selection error probability pₛₑᵤᵢₗ and threshold value Zₛₑᵤᵢₗ providing an acceptable false positive rate, false positive rate TFP being equal to:

$$TFP = \frac{{p}_{seuil} * n}{( number of genes for which {Z}_{k} \geq {Z}_{seuil} )}$$

where n is the number of considered genes.

According to an embodiment of the method of the present invention, the step of identifying the genes consists of selecting the genes having their normalized variation value greater than a first threshold value for the genes of the first group and greater than a second threshold value for the genes of the second group.

According to an embodiment of the method of the present invention, the determination of the first and second threshold values consists of selecting first and second selection error probabilities respectively desired for the first and second groups and defining the first and second corresponding threshold values by means of the cumulative calibration frequency distribution.

According to an embodiment of the method of the present invention, the selection of the first and second threshold values consists of carrying out the method of claim 4 successively for the first and the second group.

According to an embodiment of the method of the present invention, variation value Varₖ of a gene is equal to the difference between the mRNA concentrations of said gene for different cells.

According to an embodiment of the method of the present invention, variation value Varₖ of a gene is equal to the ratio of the mRNA concentrations of said gene for different cells.

According to an embodiment of the method of the present invention, the method comprises, for each list, the steps of:
classifying the genes by increasing mRNA concentrations;
assigning a zero rank value to all the genes having mRNA concentrations smaller than or equal to a threshold concentration value;
assigning a single rank value to each of the other n1 genes having an mRNA concentration greater than the threshold concentration value, the rank value ranging between 1 and n1, rank R of a gene being all the higher as the mRNA concentration of said gene is high; and
normalizing the rank values over a range from 0 to w, w being a positive integer, rank r of a gene being now equal to (R*w)/n, where n is the number of studied genes.

According to an embodiment of the method of the present invention, the variation value of a gene is equal to the difference between the gene ranks for the two analyzed lists.

According to an embodiment of the method of the present invention, the normalized variation value Z of each gene is obtained according to the following formula:

$$Z = \frac{Var - μ ( g )}{σ ( g )}$$

where Var is the variation value of said gene and μ(g) and σ(g) respectively are the average and the standard deviation of a set of variation values corresponding to a set of genes having mRNA concentrations close to the mRNA concentration of said gene.

According to an embodiment of the method of the present invention, the normalized variation value is calculated according to the steps of:
assigning a single rank value r to each gene equal to the rank value of the reference list for the genes of the first group and equal to the rank value of the test list for the genes of the second group;
calculating the normalized variation value Zₖ of the gene according to the following formula:

$$Z = \frac{Var - μ ( r )}{σ ( r )}$$

where Var is the variation of said gene, μ(r) and σ(r) respectively are the average and the standard deviation of a set of variation values corresponding to a set of genes having ranks close to the rank r of said gene.

According to a variation of the method of the present invention, the method aims at analyzing the mRNA concentration variations of a set of genes based on m identical so-called reference cell groups (GR₁ to GRₘ) and q identical so-called test cell groups (GT₁ to GT_{q}), the method comprising the steps of:
for all or part of the group combinations (C_{i,j}) comprising a reference group (GRᵢ) and a test group (GTⱼ), performing the three steps of:
   building the cumulative distribution of so-called calibration frequencies according to the method of steps h) to k) based on first and second calibration groups (GR_{étal,1} and GR_{étal,2}) both taken from among the m reference groups or both taken from among the q test groups, one of the groups being possibly the reference group (GRᵢ) or the test group (GTⱼ) of the considered group combination;
   implementing steps a) to f) to determine a normalized variation value (Z_{i,j,k}) for each gene;
   defining for each gene a so-called error probability value (p_{i,j,k}) corresponding to the normalized variation value of this gene (Z_{i,j,k}) based on the cumulative calibration frequency distribution;
calculating for each gene a regrouping value (Rₖ) according to a regrouping method taking into account all the error probabilities (p_{i,j,k}) of said gene obtained for each of the combinations (C_{i,j}) of selected reference and test groups; and
identifying as exhibiting significant mRNA concentration variations the genes having a regrouping value greater than a determined threshold regrouping value (Rₛₑᵤᵢₗ).

According to an embodiment of the previously-described method, the first and second calibration groups (GR_{étal,1} and GR_{étal,2}) are identical whatever the considered group combination.

According to an embodiment of the method of the present invention, the normalized calibration variation values (Z_{ref,k}) are calculated according to the previously-defined method

$$Z = \frac{Var - μ ( g )}{σ ( g )}$$

and the normalized variation values between a test and a reference lists are calculated according to the following formula:

$$Z = \frac{Var - {μ}_{étal} ( r )}{{σ}_{étal} ( r )}$$

where functions μ_{étal}(r) and σ_{étal}(r) are obtained by smoothing of averages μ(r) and of standard deviations σ(r) calculated prior to the normalized calibration variation values.

According to an embodiment of the present invention, the determination of the threshold regrouping value (Rₛₑᵤᵢₗ) comprises the steps of:
calculating for each gene a calibration regrouping value (R_{étal,k}) according to the regrouping method based on the calibration error probabilities (p_{étal,k}) of said gene obtained from the cumulative calibration frequency distributions calculated for each selected group combination (C_{i,j});
constructing the so-called regrouping frequency distribution based on calibration regrouping values by associating with each calibration regrouping value a so-called calibration regrouping error probability, for calibration regrouping values greater than the considered calibration regrouping value to exist;
selecting the desired selection regrouping error probability (P2ₛₑᵤᵢₗ); and
defining the threshold regrouping value (Rₛₑᵤᵢₗ) corresponding to the selection regrouping error probability (p2ₛₑᵤᵢₗ) by means of the cumulative regrouping frequency distribution.

According to an embodiment of the present invention, the step of selecting a selection regrouping error probability (p1ₛₑᵤᵢₗ) comprises the steps of:
defining the maximum false positive rate acceptable for the gene identification; and
identifying the maximum selection regrouping error probability p2ₛₑᵤᵢₗ and threshold regrouping value Zₛₑᵤᵢₗ enabling obtaining an acceptable false positive rate, the false positive rate TFP being equal to

$$TFP = \frac{{p}_{2 seuil} * n}{( number of genes for which {R}_{k} \geq {R}_{seuil} )}$$

where n is the number of considered genes.

According to an embodiment of the present invention, the regrouping method comprises the steps of:
distributing the group combinations in different sets;
calculating for each set an intermediary value for each gene equal to the product or to the sum of the error probabilities (p_{i,j,k}) of the gene obtained for each of the group combinations of the set;
calculating for each gene a regrouping value (Rₖ) equal to the average of the intermediary values calculated for each set.

According to a variation of the method of the present invention, the method aims at analyzing the variations of the mRNA concentrations of a set of genes based on m identical groups of so-called reference cell (GR₁ to GTₘ) and q identical groups of so-called test cells (GT₁ to GT_{q}), the method comprising the steps of:
carrying out steps a) and b) for each of the reference and test groups providing m reference lists and q test lists;
defining for each of the lists a rank value for each gene according to the previously-described method;
defining a global reference list associating with each gene a single rank equal to the average of its ranks in the reference lists;
defining a global test list associating with each gene a single rank equal to the average of its ranks in the test lists;
carrying out steps c) to g) from the global reference and test lists, the variation values being equal to the rank difference and the normalized variation values being calculated according to one of the previously-described methods.

According to an embodiment of the method of the present invention, one or several reference, test, or calibration lists are obtained according to a method for creating an artificial data set comprising the steps of:
implementing steps h) to k) providing a cumulative calibration frequency distribution;
defining for each gene a normalized variation value by performing a random drawing from the cumulative calibration frequency distribution, the set of the normalized variation values thus defined having a cumulative frequency distribution identical to the calibration frequency distribution.

The foregoing and other objects, features, and advantages of the present invention will be discussed in detail in the following non-limiting description of specific embodiments in connection with the accompanying drawings, among which:

FIG. 1 shows a DNA chip;

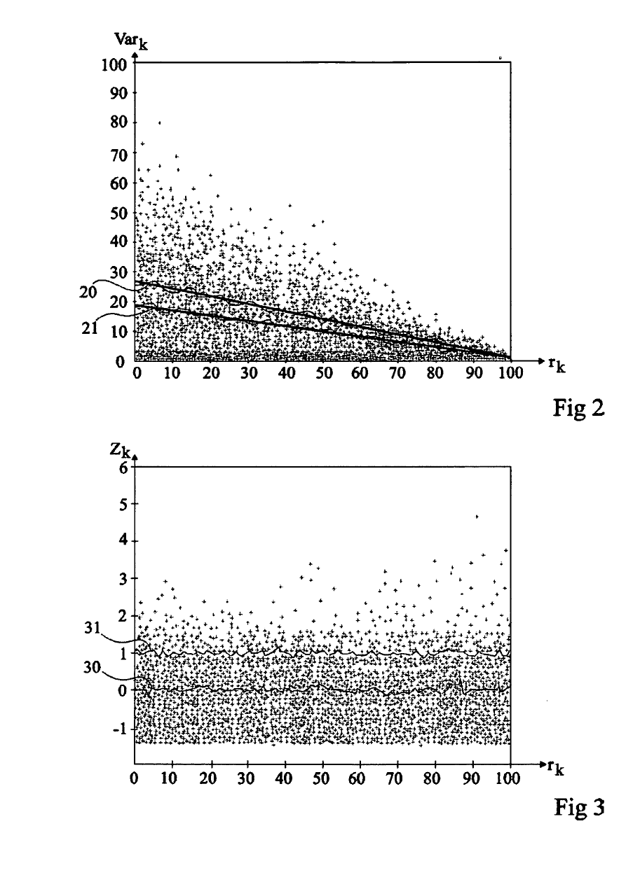

FIG. 2 is a representation of mRNA concentration variation values relative to a set of genes used according to a first step of the invention;

FIG. 3 is a representation of normalized mRNA concentration variation values relative to a set of genes used according to a second step of the invention;

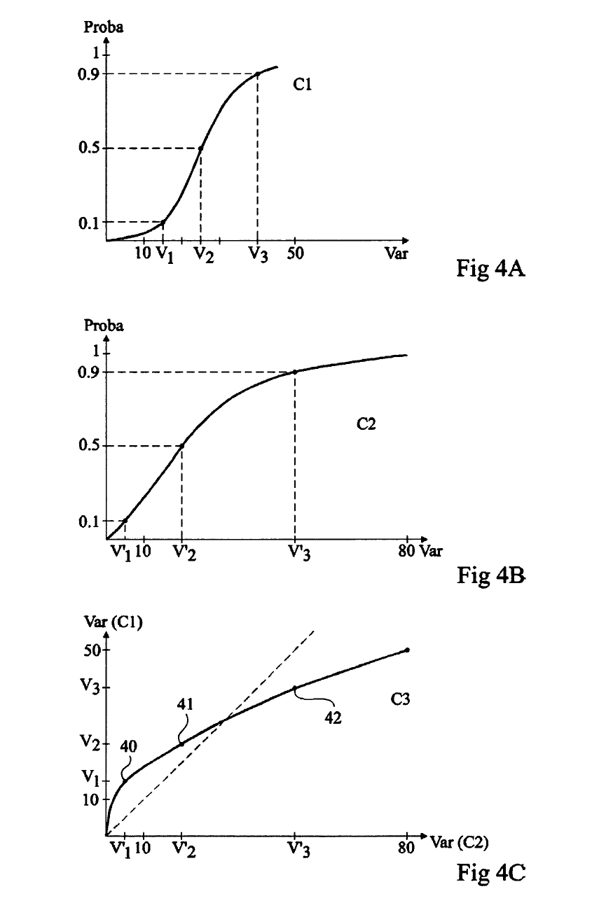

FIG. 4A shows a cumulative mRNA concentration variation value frequency distribution for a first set of genes;

FIG. 4B shows a cumulative mRNA concentration variation value frequency distribution for a second set of genes;

FIG. 4C is a “quantile versus quantile” curve of the mRNA concentration variation values of the first and second sets of genes;

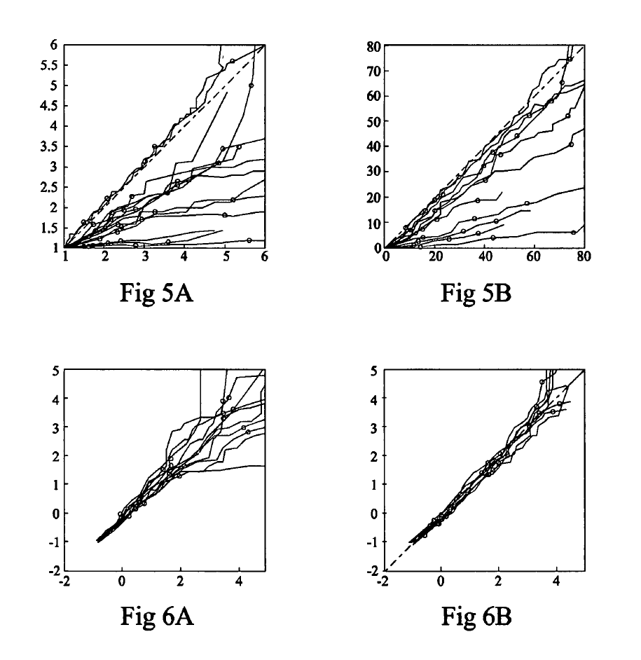

FIG. 5A shows a set of “quantile versus quantile” curves of non-normalized variation values obtained according to a fold change method;

FIG. 5B shows a set of “quantile versus quantile” curves of non-normalized variation values obtained according to a rank shift method;

FIG. 6A shows a set of “quantile versus quantile” curves of normalized variation values obtained according to a fold change method; and

FIG. 6B shows a set of “quantile versus quantile” curves of normalized variation values obtained according to a rank shift method.

The analysis method of the present invention provides analyzing by means of DNA chips a set of n genes and studying the variations of the mRNA concentrations between reference cells and test cells.

In a first part, an analysis of the variations between a test cell group and a reference cell group will be described.

In a second part, a way to determine a threshold value which enables selecting genes having significant variation values will be described.

In a third part, the advantages of the invention over prior art will be demonstrated.

In a fourth part, the method according to the invention will be generalized to the analysis of several test or reference cell groups.

In a fifth part, a method for constructing artificial data sets will be described.

In a sixth part, an application of the method according to the invention consisting of analyzing the mRNA concentration variations along time (kinetic study) or according to successive modifications of the culture conditions of a set of cells (experiment of dose/response type) will be described.

1. Comparison between a Test Group and a Reference Group

The analysis method of the present invention provides analyzing by means of DNA chips a set of n genes and studying the mRNA concentration variations between a group of reference cells and a group of test cells. The mRNA concentration cₖ relative to each gene gₖ (k being a number ranging between 1 and n) is previously measured and the values are written in reference and test lists L_{ref} and Lₜₑₛₜ.

The analysis method starts with the calculation for each of the genes of an mRNA concentration variation value, or variation value Varₖ, which may be equal to the mRNA concentration difference between the reference and test groups (Varₖ=c_{k,test}-c_{k,ref}, where c_{k,test} and c_{k,ref} respectively are the mRNA concentrations of gene gₖ on the test and reference lists) or else equal to the ratio of the mRNA concentrations (Varₖ=c_{k,test}/c_{k,ref}), which corresponds to the previously-described “fold change” method.

According to the present invention and prior to the calculation of the variation values, the genes are classified by increasing mRNA concentrations for each of the reference and test lists. A zero rank value is then assigned to all genes having an mRNA concentration equal to zero or more widely to all genes having an mRNA concentration smaller than a threshold concentration corresponding to an estimate of the measurement noise. A single rank value is then assigned to each of the n1 other genes, the rank value ranging between 1 and n1. The set of rank values forms a continuous series of integers between 0 and n1. The rank of a gene is all the higher as its mRNA concentration is high.

Further, the variations of the method of mRNA concentration measurement based on DNA chips cause a more or less significant variation in the RNA concentration values. Two identical cell groups may have concentration values ranging between 10 and 10,000 for the first group and between 50 and 11,000 for the second group.

To realign the ranges of mRNA concentration values and get rid of the possible differences between numbers n₁ of genes for which the mRNA concentration is greater than a given threshold concentration value, the rank values are normalized over a range for example from 0 to 100. Rank rₖ of a gene gₖ is now equal to (Rₖ×100)/n, where Rₖ is the non-normalized rank of gene gₖ.

According to the present invention, the variation value of each gene is expressed as being equal to the difference between the gene rank in the reference list and the gene rank in the test list. Variation value, Varₖ, of each gene gₖ, is calculated as follows:
Varₖ=r_{test,k}−r_{ref,k} (1)
where r_{test,k} and r_{ref,k} respectively are the ranks of gene gk of the test and reference lists.

This way of expressing the variation values according to the invention is called hereafter the “rank-shift” method.

FIG. 2 shows a set of positive variation values Vark calculated according to the “rank shift” method. The ranks are indicated in abscissas. The variations are indicated in ordinates. Each variation value of a gene is represented by a cross having its abscissa corresponding to the rank of this gene for the reference list. Although this is little visible in FIG. 2 due to the large considered number of genes, each abscissa value (rank) corresponds to a single gene and thus to a single variation value.

It should be noted that genes having a low rank exhibit a greater average variation amplitude than genes having a high rank value. This corresponds, as indicated previously, to that fact that, for weakly expressed genes, variations are likely to be greater. Thus, a method consisting as in prior art of setting an identical threshold variation value for genes with a weak expression and for genes with a strong expression would result in considering that the genes exhibiting a significant variation are the sole genes of low rank and thus with a low mRNA concentration.

To overcome this disadvantage, the present invention provides defining a threshold variation value which is a function of the gene rank. More specifically, the analysis method of the present invention comprises a normalization process.

The genes are classified in two groups. The genes having a variation value which indicates an increase in their mRNA concentrations between the reference list and the test list are placed in a first group. The others are placed in a second group and a new variation value is calculated for these genes by inverting the test and reference lists.

Thus, in the case where the variation value is expressed according to the rank shift method, the genes of the first group are the nₚₒₛ genes having a positive or zero variation (r_{test,k}=>r_{ref,k} for a gene gₖ) and the genes of the second group are the n_{neg} genes having a strictly negative variation (r_{test,k}<r_{ref,k} for a gene gₖ). For each gene of the second group, a variation value Varₖ equal to the opposite of the initial value is recalculated. All variation values are now positive.

In the case where the variation value is expressed according to the “fold change” method, the variation values of the genes exhibiting a decrease in their concentration (value smaller than 1) between the reference group and the test group are replaced with the inverse of the initial values. The variation values are thus all greater than 1.

According to an implementation mode of the normalization method of the present invention, a set of neighboring ranks, or rank “window”, is selected for each gene gₖ of rank rₖ. The average value of the variation values corresponding to this rank window is then calculated, to form a local average μ(gₖ).

A local standard deviation σ(gₖ) of the variation values is then calculated for each gene gₖ by using the same window as for the local average calculation.

Curves 20 and 21 of FIG. 2 respectively show the general shape of values μ(gₖ) and σ(gₖ) after smoothing.

Based on values μ(gₖ) and σ(gₖ), preferably taken after smoothing, a normalized variation value Zₖ is calculated for each of genes gₖ according to the following formula:

$${Z}_{k} = \frac{{Var}_{k} - μ ( {g}_{k} )}{σ ( {g}_{k} )}$$

According to an alternative embodiment of the method of the present invention, the normalization method is carried out separately for each of the first and second genes groups. Values μ(gₖ) and σ(gₖ) are calculated for each group based on the variation values of a set of genes of a same group.

FIG. 3 shows the set of normalized variation values Zₖ obtained for each of variation values Varₖ of FIG. 2. As in FIG. 2, the abscissas designate the ranks and an abscissa value corresponds to a single normalized variation value. Curves 30 and 31 respectively correspond to the local averages and to the local standard deviations, non smoothed, calculated based on values Zₖ in the same way as was previously done based on values Varₖ, as described hereabove. Curves 30 and 31 show that the local averages and the local standard deviations now are substantially constant whatever the rank, which means that the genes having different average mRNA concentrations have normalized variation values which follow the same cumulative frequency distribution.

Generally, any normalization method such that the cumulative frequency distribution of a subset of normalized variation values corresponding to genes of a same rank window is substantially identical whatever the considered subset may be used.

At the end of the normalization step, a threshold value Zₛₑᵤᵢₗ is determined, which may be different for the first and for the second gene groups, and the genes having a normalized deviation value exceeding the threshold value are selected.

According to a major aspect of the present invention, this threshold value is identical for all genes and the selection criterion is hanogenous whatever the rank of the analyzed genes, that is, independently from their average mRNA concentration.

An advantage of the analysis method according to the present invention is that it enables identifying genes exhibiting a significant variation in their mRNA concentration based on a reduced number of measurements.

2. Determination of a Threshold Value

The present invention also provides defining a threshold value according to the following method.

A calibration step consisting of determining the variations of the normal mRNA concentrations of each of the genes by studying two so-called calibration identical cell groups is then performed, the mRNA concentration of each gene being written in the two sampling lists L_{étal,1} and L_{étal,2}.

A calculation of calibration variation values normalized according to the previously-described rank shift method and normalization process is then performed. One of the two sampling lists L_{étal,1} and L_{étal,2} is considered as a test list while the other one is considered as a reference list. A calibration variation value Var_{étal,k} is thus obtained for each gene gk and a normalized calibration variation value Z_{étal,k} is obtained for each of the genes.

A set of normalized calibration variation values having substantially constant local averages and local standard deviations are here again obtained.

In an embodiment of the method of the present invention, a smoothing of local averages μ_{étal}(gₖ) and of local standard deviations σ_{étal}(gₖ) used to calculate the Z_{étal,k} values is performed. Two calibration curves showing average μ_{étal}(r) and standard deviation σ_{étal}(r) of the calibration values versus the rank are obtained, any reference to a given gene being suppressed. In a coaaarison between a test group and a reference group, normalized variation values Zₖ are calculated based on these calibration curves according to the following formula:

$${Z}_{k} = \frac{{Var}_{k} - {μ}_{étal} ( {r}_{k} )}{{σ}_{étal} ( {r}_{k} )}$$

The groups of calibration cells may be reference cells, test cells, or other cells believed to be adapted. The selection of the used cells is dictated by the effect of values μ_{étal}(r) and σ_{étal}(r) on normalized variation values Zk. The latter are all the smaller as the average and standard deviation values are high. Values μ_{étal}(r) and σ_{étal}(r) depend, on the one hand, on the reproducibility of the experimental conditions (not perfectly identical DNA chips) and, on the other hand, on the stability of the biological system of the selected cells. The experimental conditions being assumed to be reproducible, a biological system will exhibit values μ_{étal}(r) and σ_{étal}(r) which are all the higher as it is unstable. Thus, a calibration based on two cancerous cells will provide higher values μ_{étal}(r) and σ_{étal}(r), as compared to those obtained from two normal cells. Accordingly, the calibration must be performed on a biological system which has the same stability characteristics as the system formed by the test and the reference.

In the case where the test and the reference both have been duplicated, the calibration curves are constructed independently for each of the couples, which results in two couples of calibration curves (μₜₑₛₜ, σₜₑₛₜ) and (μ_{ref}, σ_{ref}). Which of the two systems is more unstable (higher μ or/and σ) is then evaluated. This evaluation may be performed in different ways. Two sets of normalized variation values may for example be calculated by respectively using (μₜₑₛₜ, σₜₑₛₜ) and (μ_{ref}, σ_{ref}). A cumulative frequency distribution may for example be constructed for each set. The two normalized variation values corresponding, for example, to the 75^{th} percentile (probability equal to 0.75) are then compared. The system having the greatest value is the most unstable. Generally, the results of the analysis method of the present invention are better if the calibration curves constructed based on the most unstable system are used.

According to an aspect of the present invention, a cumulative calibration frequency distribution is constructed based on all the normalized variation values. The normalized variation values of all genes, whatever their rank, follow this cumulative calibration frequency distribution. Indeed, as will be more specifically discussed in relation with FIG. 6B, any subset of normalized calibration variation values corresponding to genes of a same rank window follows the same cumulative frequency distribution and it is thus possible to build a single cumulative frequency distribution based on all the normalized calibration variation values. Given the large number of studied genes and thus the large number of obtained normalized calibration variation values, the resulting cumulative frequency distribution is very accurate.

Based on this cumulative calibration frequency distribution, with any normalized calibration variation value Z_{étal,k} is associated a so-called selection error probability p_{seuil,k}, for calibration variation values naturally greater than the latter to exist.

In a comparative analysis between test and reference cells according to the method previously described in relation with FIGS. 2 and 3, the selection error probability pₛₑᵤᵢₗ corresponding to the probability for normalized variation values greater than the threshold value Zₛₑᵤᵢₗ chosen to select the genes to naturally exist can now be defined by means of the cumulative calibration frequency distribution.

An advantage of the analysis method according to the present invention is that it enables associating a selection error probability with any selected threshold value Zₛₑᵤᵢₗ.

Another advantage of the analysis method according to the present invention is that it enables selecting a very accurate threshold value Zₛₑᵤᵢₗ with a reduced number of measurements.

Based on the cumulative calibration frequency distribution, it is possible to define a set of statistic parameters, their knowledge enabling best selection of selection error probability pₛₑᵤᵢₗ.

Knowing the number of studied genes, the proportion of “normal” genes among the set of genes identified as having a normalized variation value Zₖ greater than Zₛₑᵤᵢₗ can be known. This proportion of normal genes is called the false positive rate TFP and is defined as follows:

$$TFP = \frac{{p}_{seuil} * n}{( number of genes for which Z \geq {Z}_{seuil} )}$$

In the case of a distinct analysis of the first and second gene groups, a first and a second false positive rate are defined. n is replaced with the number of genes of the first group nₚₒₛ or of the second group n_{neg}, values pₛₑᵤᵢₗ/Zₛₑᵤᵢₗ being possibly different for each gene group.

A very small selection error probability pₛₑᵤᵢₗ providing a very small false positive rate may be selected. However, it may be advantageous to select a greater probability pₛₑᵤᵢₗ and thus a greater Zₛₑᵤᵢₗ to select and thus subsequently study a larger number of genes.

In addition to the false positive rate, it is possible to know the selection sensitivity. The cumulative frequency distribution of the normalized variation values Zₖ obtained in the comparison between the test and reference cells is previously constructed. Based on this distribution, it is possible to associate with any normalized variation value Zₖ a so-called observation probability p_{obs,k} for normalized values greater than the latter to be observed.

Based on the values of the selection error probability p_{seuil,k} and of the observation probability p_{obs,k} of each gene, it is possible to define fraction F of genes for which variation value Varₖ has increased with respect to calibration variation value Var_{étal,k}. Fraction F is defined as being the maximum value of the set of values p_{obs,k}-p_{seuil,k} calculated for each gene gₖ (F=max[p_{obs,k}-p_{seuil,k}]). If threshold p_{seuil,k} is the selected selection error probability, the false positive rate can be defined as being equal to p_{seuil,k}/p_{obs,k}. When a couple of values Pseuil/Zₛₑᵤᵢₗ is selected, the sensitivity, equal to (p_{obs,k}-p_{seuil,k})/F enables knowing whether among the selected genes, the number of genes really exhibiting significant variations is representative of the number of genes, the variation values of which have increased (Varₖ>Var_{étal,k}).

An advantage of the analysis method according to the present invention is that it enables associating a positive false value and a sensitivity value with any threshold value Zₛₑᵤᵢₗ and thus with any selected selection error probability pₛₑᵤᵢₗ.

3. Demonstration of the Advantages of the Present Invention

FIGS. 4A to 4C illustrate the construction of a “quantile versus quantile” curve. FIG. 4A shows a cumulative frequency distribution Cl of a first subset of variation values taken from among the set of variation values (Var) obtained in a comparative study. The variation values are plotted in abscissas. The probability (proba) for variation values smaller than the variation value in abscissas to exist is indicated in ordinates.

FIG. 4B is another cumulative frequency distribution C2 of a second set of variation values taken from among the variation values of the comparative study.

FIG. 4C is a “quantile versus quantile” curve C3 obtained from curves C1 and C2 of FIGS. 4A and 4B. The variation values of the first studied set are shown in ordinates, and the variation values of the second studied set are shown in abscissas. The “quantile versus quantile” curve is obtained by plotting for each probability value (between 0 and 1) the corresponding variation values on curves C1 and C2 and by defining a point having these two values respectively as an ordinate and an abscissa. Point 40 of curve C3 has V1′ as an abscissa and V1 as an ordinate, V1 and V1′ respectively being the variation values of curves C1 and C2 corresponding to probability 0.1. Similarly, points 41 and 42 of curve C3 have as respective abscissas V2′ and V3′ and as respective ordinates V2 and V3, variation values V2, V3 of curve C1 and V2′, V3′ of curve C2 having as respective probabilities 0.5 and 0.9. The “quantile versus quantile” curve is thus obtained for two subsets of variation values. In the example of FIG. 4C, curve C3 is relatively distant from the diagonal plotted in dotted lines, which means that the first and second subsets of variation values having different distribution functions.

FIG. 5A shows a set of “quantile versus quantile” curves obtained by studying different subsets of variation values calculated according to a fold change method. The most flattened curves are obtained by taking subsets of variation values having very distant respective ranks. This shows that genes with different ranks have variation values which follow different distribution functions.

FIG. 5B shows a same subset of “quantile versus quantile” curves obtained by studying different subsets of non-normalized variation values calculated according to a rank shift method. Here again, a difference between the distribution functions can be observed for genes having very distant ranks.

FIG. 6A shows a set of “quantile versus quantile” curves obtained by studying different subsets of normalized variation values calculated according to the fold change function and the normalization method of the present invention. The curves comes close to the diagonal, which means that genes having different ranks have normalized variation values which follow relatively similar distribution functions. However, relatively significant divergences can be observed for values corresponding to high probabilities.

FIG. 6B shows a set of “quantile versus quantile” curves obtained by studying different subsets of normalized variation values calculated according to the rank shift method and the normalization method of the present invention. The curves are all very close to the diagonal, which means that the set of normalized variation values follows the same cumulative frequency distribution.

This shows that, by combining a variation value calculation according to the rank shift method of the present invention and a normalization of these values according to the normalization method of the present invention, a set of normalized variation values which follow the same cumulative reference frequency distribution is obtained.

As a result, due to the analysis method according to the present invention, each gene can be studied individually based on three measurements only of mRNA concentrations with DNA chips while a large number of measurements was necessary before.

4. Comparison between Several Test and Reference Groups

In the case where several mRNA concentration measurements for each gene are available and obtained from m reference groups GR₁ to GRₘ and q test groups GT₁ to GT_{q}, a multiple analysis method according to the present invention enables finer identification of which genes exhibit the more significant mRNA concentrations.

The multiple analysis method comprises multiple analyses of the variation between reference and test lists. For all or part of combinations C_{i,j} comprising a reference group GRᵢ and a test group GTⱼ, for each gene gₖ, a variation value Var_{i,j,k} is calculated according to the rank shift method and a normalized variation value Z_{i,j,k} is calculated according to the normalization method of the present invention.

In parallel, a calibration step identical to that described previously is carried out. After selection of two calibration groups GR_{étal,1} and GR_{étal,2} from along the m reference groups, a normalized calibration variation value Z_{étal,k} is calculated for each gene gₖ by means of the rank shift method and the normalization method of the present invention. A cumulative frequency distribution is constructed from all the normalized calibration variation values. It is thus possible to associate with a calibration normalized variation value Z_{étal,k} a so-called calibration error probability p_{étal,k} for normalized variation values naturally greater than the latter to exist.

According to an alternative embodiment, a regrouping cumulative frequency distribution is built for each selected combination C_{i,j} from the two reference groups, one of which is group GRᵢ, or of two test groups, one of which is group GTⱼ of the considered combination C_{i,j}.

Based on the cumulative calibration frequency distributions, a so-called error probability p_{i,j,k} is defined for each gene gₖ, corresponding to the normalized variation value Z_{i,j,k} of said gene. In the case where a single cumulative calibration frequency distribution is available, error probabilities p_{i,j,k} are all equal.

According to an alternative embodiment, it is determined whether the variation values of a gene obtained for each combination C_{i,j} correspond to an increase (positive variation) or to a decrease (negative variation) of the mRNA concentrations between reference cell group GRᵢ and test cell group GTⱼ. For a specific gene gₖ, some of probabilities p_{i,j,k} correspond to positive variations and other values p_{k,l} correspond to negative variations. Product Prodpₚₒₛ of the values p_{i,j,k} corresponding to positive variations is compared with product Prodp_{neg} of the values p_{i,j,k} corresponding to negative values. If Prodₚₒₛ is smaller than Prod_{neg}, the gene variation is considered as positive and all the probabilities p_{i,j,k} corresponding to negative variations take value 1 (and conversely, if Prodₚₒₛ>Prod_{neg}, the gene variation is considered as negative and all probabilities p_{i,j,k} take value 1). Generally, the result is homogeneous, that is, the variation of gene k is considered as positive (or negative) for all combinations. If, for a minority of sets, the assignment procedure has resulted in providing gene gₖ with an opposite variation direction, this can be explained by the presence of an abnormal, so-called artifactitious variation, which can be easily spotted. Such values are eliminated, which results in a correct reassignment of the variation direction.

A regrouping value Rₖ is then calculated according to a regrouping method for each gene gₖ based on the error probabilities of the gene. According to the same method, a calibration regrouping value R_{étal,k} is calculated for each gene gₖ by using the calibration error probabilities p_{étal,i,j,k} corresponding to the normalized variation values Z_{étal,i,j,k} of each gene obtained based on the previously-calculated cumulative frequency distributions.

According to an implementation mode of the regrouping method of the present invention, the selected combinations are distributed in different sets. Independent combinations may for example be formed, two combinations C_{i1,j1} and C_{i2,j2} being independent if groups GRᵢ₁ and GRᵢ₂ are different and if groups GTⱼ₁ and GTⱼ₂ are different. In the case where there are as many reference groups as there are test groups (m=q), m! sets of m independent combinations may for example be formed (if m<q, q!/m! sets of m independent comparisons may be formed). The product (or the sum) of all the error probabilities P_{i,j,k} of a same gene gₖ in each set is then calculated for each set and an intermediary value is obtained for each set. A regrouping value Rₖ is then calculated for each gene gₖ by taking the average of the intermediary values of each set.

As for a simple analysis between a reference list and a test list, a threshold regrouping value Rₛₑᵤᵢₗ is defined to select the genes exhibiting regrouping values greater than the latter. For this purpose, a so-called regrouping cumulative frequency distribution is constructed from all the calibration regrouping values. To any regrouping value Rₖ corresponds a so-called theoretical probability P_{theo,k}, for regrouping values greater than Rₖ to exist. A regrouping selection probability p₂ₛₑᵤᵢₗ can then be associated with any selected threshold regrouping value Rₛₑᵤᵢₗ. Rₛₑᵤᵢₗ and pₛₑᵤᵢₗ will be selected according to the false positive rate and to the desired sensitivity.

This multiple analysis process enables increasing the analysis power since it enables selecting genes having small mRNA concentration variations, non-significant in all individually-taken comparisons, but which become significant when all possible comparisons are taken into account.

b. Average Analysis

The method of multiple analysis by analysis of averages consists of constructing for groups GR₁ to GRₘ and GT₁ to GT_{q} a single group GR and GT. The mRNA concentration values of groups GR₁ to GRₘ and GT₁ to GT_{q} are expressed in the form of rank values, normalized over a scale from 0 to 100, as described in chapter 1. Two new lists Lₜₑₛₜ and L_{ref} indicating for each gene a single rank value equal to the average of the rank values respectively of the test groups and of the reference groups are constructed.

Two calibration lists L_{étal1,k} and L_{étal2,k} are then built based on two sets of N identical cell groups (reference, test, or other), with N=m if m<=q, or N=p if p<=m, according to the previously-described method. The same analysis method as that implemented in a comparison between a single test group and a single reference group is then carried out, the cumulative calibration frequency distribution being constructed from two calibration lists L_{étal1,k} and L_{étal2,k}.

5. Construction of an Artificial Data Set

According to an aspect of the present invention, the cumulative frequency distribution of the normalized transcription signal variations for a biological system enables constructing artificial sets of data, in the form of an artificial list Lₐᵣₜ associating with each gene a concentration value, the data set having the same statistic features as the real data having been used for the calibration.

Based on two identical cell groups G1 and G2, the smoothed calibration curves μ_{étal}(gₖ) and σ_{étal}(gₖ), as well as the cumulative frequency distribution of the normalized calibration variation values, are constructed as described hereabove.

An artificial data set is then constructed indifferently exclusively from G1 or from G2 or from G1 and G2, used in turns. If, for example, G1 is taken as a base to artificially generate a data set, rank rₖ of gene gₖ is considered.

A number is randomly drawn from a linear distribution over interval [0,1]. By interpolating this number over the cumulative calibration frequency distribution, a normalized variation value Zₖ is drawn for gene gₖ. If gene gₖ increases between G1 and G2, this normalized variation value is turned into a variation value according to the following formula:
Varₖ=Zₖ*σ_{étal}(rₖ)+σ_{étal}(rₖ)
and the new rank, r_{jeu,k}, of gene gₖ, is deduced therefrom by formula r_{jeu,k}=rₖ+Varₖ.

If r_{jeu,k} is greater than 100, it is given value 100. If gene gₖ decreases between G1 and G2, the new rank r_{jeu,k} must be found, such that:
Varₖ=Zₖ*σ_{étal}(r_{jeu,k})+μ_{étal}(r_{jeu,k}) and r_{jeu,k}=rₖ−Varₖ±εᵣ, where εᵣ is a constant to be determined.

One possibility to search for r_{jeu,k} consists of successively calculating, starting from the value just under rₖ, the absolute value of Er for any value r_{jeu,k} smaller than rₖ and of taking as a new rank, the rank r_{jeu,k} for which the absolute value of εᵣ reaches the first local minimum (that is, when the absolute value of εᵣ at the rank just under the considered r_{jeu,k} becomes greater than at rank r_{jeu,k}).

If rank zero is reached without fulfilling the second condition, r_{jeu,k} is chosen to be equal to zero.

The new set of values thus obtained may be easily transformed into mRNA concentration values by the transformation inverse to that providing the rank. The mRNA concentration of each gene is written on artificial list Lₐᵣₜ.

It is possible to generate several artificial lists according to the above-described method. These lists can be used in a comparison between several test and reference cell groups, especially when the number of test groups and the number of reference groups differ. Generally, an artificial data set may replace any group of cells used in the previously-described analyses.

6. Kinetic or Dose/Response Experiment Analysis

In the case where several measurements of the transcription activity are available and obtained from several n+1 sets of the groups, n being an integer. First group GC0 contains i₀ groups GC0₁ to GC0ᵢ₀, second group GC1 contains i₁ groups GC1₁ to GC1ᵢ₁, last group GCn contains iₙ groups GCn₁ to GCnᵢₙ. A multiple method according to the present invention enables finer identification of the genes exhibiting the most significant transcription variations. Groups GC1 to GCn may represent measurements performed on the same biological system, but at different and increasing times (kinetic experiment), or submitted to a stimulus of strictly increasing or decreasing intensity (dose/response experiments). The common feature of these two types of experiments is that it is searched, for each gene gₖ, whether a significant transcription signal variation has occurred over the entire interval of independent variable VI (time, in the case of a kinetic experiment, or dose of a product in the case of a dose/response). The values of the independent variable are arbitrarily taken to be equal to VI=0, 1, . . . n.

In a first phase of the analysis, all the analyses concerning the groups for which VI=i and VI=i+1 are independently carried out, according to the above-described methods. For example, one of the analyses will bear on groups GC0 and GC1, another one on groups GC1 and GC2, and the last one will bear an groups GCn−1 and GCn. For each analysis and for each gene, the values of p_{theor,k} (or p_{seuil,k} if there is a single group) and of P_{obs,k} are determined. The genes having undergone a significant mRNA concentration variation are selected by means of the selection parameters such as the regrouping selection error probability, the false positive rate, or yet the sensitivity. For each gene, a sequence of ordered results S_{sens,k} which indicates for each interval VI whether the gene has or not been detected as non-varying or positively or negatively varying, and another sequence of ordered results, S_{sel,k}, which indicates whether the variation is significant, are then obtained. Thus, for gene gₖ, there could be sequence S_{sens, k}=+,+,0,−,−,−,+,+ and sequence S_{sel,k}=1,1,0,0,0,0,0,0. It should be noted that here, as in the following, a position for which no variation has been detected (0 in S_{sens,k}) still remains at zero in S_{sel,k}.

Then, if there exists at least one gene gᵢ for which there is a zero at two consecutive positions of S_{sel,i}, without for a zero to be at one of the corresponding positions in S_{sens,i}, all the analyses concerning the groups for which VI=i and VI=i+2, and for which there exist genes such as gene gᵢ, are performed independently according to the above-described methods. For example, one of the analyses will bear on groups GC0 and GC2, another one will bear on groups GC1 and GC3, and the last one will bear on groups GCn−2 and GCn. Similarly, the genes having undergone a significant variation are selected. List S_{sens,k} is not modified. List S_{sel,k} is completed as follows: if a significant variation has been detected between values i and i+2 of VI, and if positions i and i+1 were at zero at the preceding step, then positions i and i+1 are changed to one. If one of the positions was already at one, the new result is not considered as significant as concerns the second position. Thus, the new sequence for k might by S_{sel,k}=1,1,0,1,1,1,0,0. Positions 4, 5, and 6 have been set to 1, since the analysis bearing on the groups corresponding to VI=3 and VI=5, as well as the analysis bearing on the groups corresponding to VI=4 and VI=6, have resulted in the selection of gene gₖ.

The analysis carries on at the orders of greater degrees, such as the order of degree 3 (VI=i and VI=i+3), etc. as long as necessary (existence of at least one gene i, having a sequence of zeroes of same degree in S_{sel,i} and no zero in one of the corresponding positions in S_{sens,i})

At the end of the analysis process, all the genes having at least one position set to one in Sₛₑₗ are selected. This procedure enables efficiently filtering the genes which have shown a significant variation in an interval of contiguous values of VI. These genes may then be more finely regrouped by a regrouping method.

An additional selection and a first qualitative regrouping of the variation curves according to VI can then be performed by applying sequence S_{sel,k} on sequence S_{sens,k} as follows: for any position of S_{sel,k} equal to one, the values at the corresponding positions of S_{sel,k} are kept, and for any position of S_{sel,k} equal to zero, the values at the corresponding positions of S_{sel,k} are placed in brackets. Thus, S_{sel,k}=1,1,0,1,1,1,0,0 and S_{sens,k}=+,+,0,−,−,++ will provide, S_{sens,k}=+,+,(0),−,−,−,(+),(+).

This representation enables additional selection based on simple criteria. For example, in a dose/response experiment, it can be imposed, as an additional condition, that the variation be monotonous. In this case, gene gₖ such that S_{sens,k}=+,+,(0),−,−,−,(+),(+) would not be retained. However, gene gⱼ such that S_{sens,j}=+,+,(+),(0),(−),+,(+),(+) would be retained, since all the significant variations are positive. Similarly, if biological or other arguments enable believing that starting, for example, from the fourth value of VI (marked with a | hereafter), there must be a change in the variation sense, gene l such that S_{sens,l}=+,+,(+),|(−),(−),−,(+),− would be kept and gene m such that S_{sens,m}=−,−,(+),|(+),(+),−,(−),− would be eliminated.

This representation also enables fast regrouping of the mRNA concentration signal profiles which are comparable. For example, the genes such that S_{sens,n}=+,+,(+),(−),(−),−,(+), and such that S_{sens,o}=+,+,(+),(+),(+),−,(−),−, which have significant positive variations at the same positions 1 and 2 and significant negative variations at the same positions 6 and 8 will be regrouped.

Of course, the present invention is likely to have various alterations and modifications which will readily occur to those skilled in the art. In particular, the method of the present invention may apply to the analysis of the variations of the number of different proteins present in living cells.

Further, the analysis method of the present invention may be implemented from mRNA concentrations noted for each of the studied gene sequences corresponding to a hybridization unit of the used DNA chip. Not the variations of the mRNA concentration relative to a gene, but that relative to a given sequence, will thus be studied.

Moreover, a different definition of the variation values may be used. Similarly, other normalization processes fulfilling the requirement of uniformity of the cumulative frequency distributions of any subset of normalized variation values may be provided. Further, it will be within the abilities of those skilled in the art to define the optimal regrouping process enabling identification of the genes exhibiting the most significant mRNA concentration variation values.

1. A method for analyzing the variations of concentrations of messenger RNAs obtained by transcription of a set of genes, comprising the steps of:
a) measuring the messenger RNA concentration for each of the genes in so-called reference cells and writing the results in a reference list (L_{ref});
b) measuring the messenger RNA concentration for each of the genes in so-called test cells and writing the results in a test list (Lₜₑₛₜ);
c) calculating for each gene a variation value (Varₖ), k being an integer ranging between 1 and n, which is a measurement of the difference between the mRNA concentrations of said gene between the reference list (L_{ref}) and the test list (Lₜₑₛₜ);
d) classifying the genes in first and second groups, according to whether the genes have variation values respectively corresponding to an increase or to a decrease in their IIk concentrations between the reference list and the test list;
e) calculating for each gene of the second group a new variation value (Varₖ) which is a measurement of the difference between the mRNA concentrations of said gene between the test list and the reference list;
f) calculating for each gene a normalized variation value (Zₖ) such that the cumulative frequency distribution of a subset of normalized variation values corresponding to genes having close mRNA concentrations is identical whatever the considered subset;
g) identifying the genes exhibiting significant mRNA concentration variations based on the normalized variation values.

2. The method of claim 1, in which the step of identifying the genes consists of selecting the genes having a normalized variation value greater than a determined threshold value (Zₛₑᵤᵢₗ).

3. The method of claim 2, in which the determination of the threshold value (Zₛₑᵤᵢₗ) comprises the steps of:
h) measuring the mRNA concentration for each of the genes of two identical so-called calibration cell groups and writing the respective results in a first (L_{étal,1}) and second (L_{étal,2}) sampling lists;
i) calculating for each gene a calibration variation value (Var_{étal,k}) according to the method of steps c) to e) based on the first (L_{étal,1}) and second (L_{étal,2}) sampling lists;
j) calculating for each gene a normalized calibration variation value (Z_{ref,k}) according to the method of step f);
k) constructing the so-called calibration cumulative frequency distribution of the normalized calibration variation values associating with each normalized calibration variation value (Z_{ref,k}) a so-called selection error probability (P_{seuil,k}) for normalized calibration variation values greater than the considered normalized variation value to exist;
l) selecting the desired selection error probability (pₛₑᵤᵢₗ); and
m) defining the threshold value (Zₛₑᵤᵢₗ) corresponding to the desired selection error probability (pₛₑᵤᵢₗ) by means of the cumulative calibration frequency distribution.

4. The method of claim 3, in which the step of selecting the selection error probability (pₛₑᵤᵢₗ) comprises the steps of:
defining the maximum false positive rate acceptable for the gene identification; and
identifying the maximum selection error probability pₛₑᵤᵢₗ and threshold value Zₛₑᵤᵢₗ providing an acceptable false positive rate, false positive rate TFP being equal to:
$$TFP = \frac{{p}_{seuil} * n}{( number of genes for which {Z}_{k} \geq {Z}_{seuil} )}$$
where n is the number of considered genes.

5. The method of claim 1, in which the step of identifying the genes consists of selecting the genes having their normalized variation value greater than a first threshold value for the genes of the first group and greater than a second threshold value for the genes of the second group.

6. The method of claims 3 and 5, in which the determination of the first and second threshold values consists of selecting first and second selection error probabilities respectively desired for the first and second groups and defining the first and second corresponding threshold values by means of the cumulative calibration frequency distribution.

7. The method of claim 6, for which the selection of the first and second threshold values consists of carrying out the method of claim 4 successively for the first and the second group.

8. A method for analysis the mRNA concentration variations of a set of genes based on m identical groups of so-called reference cells (GR₁ to GRₘ) and q identical groups of so-called test cells (GT₁ to GT_{q}), the method comprising the steps of:
a2) measuring, for each reference group, the messenger RNA concentration for each of the genes and writing the results in m reference lists (L_{ref1} to L_{ref2});
b2) measuring, for each test group, the messenger RNA concentration for each of the genes and writing the results in q test lists (Lₜₑₛₜ₁ to Lₜₑₛₜ₂);
for all or part of the group combinations (C_{i,j}) comprising a reference group (GRᵢ) and a test group (GRⱼ), carrying out the following steps c2 to l2:
c2) calculating for each gene a variation value (Varₖ), k being an integer ranging between 1 and n, which is a measurement of the interval between the mRNA concentrations of said gene between the reference list (L_{refi}) and the test list (Lₜₑₛₜⱼ);
d2) classifying the genes in first and second groups, according to whether the genes exhibit variation values respectively corresponding to an increase or to a decrease in their mRNA concentrations between the reference list (L_{refi}) and the test list (Lₜₑₛₜⱼ);
e2) calculating for each gene of the second group a new variation value (Var_{i,j,k}) which is a measurement of the interval between the mRNA concentrations of said gene between the test list (Lₜₑₛₜⱼ) and the reference list (L_{refi});
f2) calculating for each gene a normalized variation value (Z_{i,j,k}) such that the cumulative frequency distribution of a subset of normalized variation values corresponding to genes having close mRNA concentrations is identical whatever the considered subset;
h2) selecting first and second calibration groups (GR_{étal,1,i,j} and GR_{étal,2,i,j}) both taken from among the m reference groups or both taken from among the q test groups, one of the groups possibly being the reference group (GRᵢ) or the test group (GTⱼ) of the considered group combination;
i2) calculating for each gene a calibration variation value (Var_{étal,i,j,k}) according to the method of steps c2) to e2) based on first (L_{étal,1,j,k}) and second (L_{étal,2,j,k}) calibration lists corresponding to the first and second calibration groups;
j2) calculating for each gene a normalized calibration value (Z_{ref,i,j,k}) according to the method of step f2);
k2) constructing the cumulative so-called calibration frequency distribution of the normalized calibration variation values associating with any normalized calibration variation value (Z_{ref,i,j,k}) a so-called selection error probability (P_{seuil,i,j,k}) for normalized calibration variation values greater than the considered normalized variation value to exist;
l2) defining for each gene a so-called error probability value (p_{i,j,k}) corresponding to the normalized variation value of this gene (Z_{i,j,k}) based on the cumulative calibration frequency distribution;
m2) calculating for each gene a regrouping value (Rₖ) according to a regrouping method taking into account all the error probabilities (p_{i,j,k}) of said gene obtained for each of the combinations (C_{i,j}) of selected reference and test groups; and
n2) identifying as exhibiting significant mRNA concentration variations the genes having their regrouping value greater than a determined threshold regrouping value (Rₛₑᵤᵢₗ).

9. The method of claim 8, in which the first and second calibration groups (GR_{étal,1} and GR_{étal,2}) are identical whatever the considered group combination.

10. The method of claim 8 or 9, in which the determination of the threshold regrouping value (Rₛₑᵤᵢₗ) comprises the steps of:
calculating for each gene a calibration regrouping value (R_{étal,k}) according to the regrouping method based on the calibration error probabilities (P_{étal,k}) of said gene obtained from the cumulative calibration frequency distributions calculated for each selected group combination (C_{i,j});
building the so-called regrouping cumulative frequency distribution based on the calibration regrouping values by associating with each calibration regrouping value a so-called calibration regrouping error probability for calibration regrouping values greater than the considered calibration regrouping value to exist;
selecting the desired selection regrouping error probability (p2ₛₑᵤᵢₗ); and
defining the threshold regrouping value (Rₛₑᵤᵢₗ) corresponding to the selection regrouping error probability (p2ₛₑᵤᵢₗ) by means of the cumulative regrouping frequency distribution.

11. The method of claim 10, in which the step of selecting a selection regrouping error probability (p2ₛₑᵤᵢₗ) comprises the steps of:
defining the maximum false positive rate acceptable for the gene identification; and
identifying the maximum selection regrouping error probability p2ₛₑᵤᵢₗ and threshold regrouping value Zₛₑᵤᵢₗ providing an acceptable false positive rate, false positive rate TFP being equal to
$$TFP = \frac{{p}_{2 seuil} * n}{( number of genes for which {R}_{k} \geq {R}_{seuil} )}$$
where n is the number of considered genes.

12. The method of claim 8, in which the regrouping method comprises the steps of:
distributing the group combinations in different sets;
calculating for each set an intermediary value for each gene equal to the product or to the sum of the error probabilities (p_{i,j,k}) of the gene obtained for each of the group combinations of the set;
calculating for each gene a regrouping value (Rₖ) equal to the average of the intermediary values calculated for each set.

13. The method of claim 1 or 8, in which the variation value (Varₖ) of a gene is equal to the difference between the mRNA concentrations of said gene for different cells.

14. The method of claim 1 or 8, in which the variation value (Varₖ) of a gene is equal to the ratio of the mRNA concentrations of said gene for different cells.

15. The method of claim 1 or 8, comprising, for each list, the steps of:
classifying the genes by increasing mRNA concentrations;
assigning a zero rank value to all the genes having mRNA concentrations smaller than or equal to a threshold concentration value;
assigning a single rank value to each of the other n1 genes having an mRNA concentration greater than the threshold concentration value, the rank value ranging between 1 and n1, rank R of a gene being all the higher as the mRNA concentration of said gene is high; and
normalizing the rank values over a range from 0 to w, w being a positive integer, rank r of a gene being now equal to (R*w)/n, where n is the number of studied genes.

16. The method of claim 15, in which the variation value of a gene is equal to the difference between the gene ranks for the two analyzed lists.

17. The method of claim 1 or 8, in which the normalized variation value Z of each gene is obtained according to the following formula:
$$Z = \frac{Var - μ ( g )}{σ ( g )}$$
where Var is the variation value of said gene and μ(g) and σ(g) respectively are the average and the standard deviation of a set of variation values corresponding to a set of genes having mRNA concentrations close to the mRNA concentration of said gene.

18. The method of claim 1 or 8, in which the normalized variation value is calculated according to the steps of:
assigning a single rank value r to each gene equal to the rank value of the reference list for the genes of the first group and equal to the rank value of the test list for the genes of the second group;
calculating the normalized variation value Z of the gene according to the following formula:
$$Z = \frac{Var - μ ( r )}{σ ( r )}$$
where Var is the variation of said gene, μ(r) and σ(r) respectively are the average and the standard deviation of a set of variation values corresponding to a set of genes having ranks close to rank r of said gene.

19. The method of claim 3 or 8, in which the normalized calibration variation values (Z_{ref,k}) are calculated according to the following method:
assigning a single rank value r to each gene equal to the rank value of the reference list for genes of the first group and equal to the rank value of the test list for genes of the second group;
calculating normalized calibration variation value Z of the gene according to the following formula:
$$Z = \frac{Var - μ ( r )}{σ ( r )}$$
where Var is the calibration variation of said gene, μ(r) and σ(r) respectively are the average and the standard deviation of a set of calibration variation values corresponding to a set of genes having ranks close to rank r of said gene,
and in which the normalized variation values between a test list and a reference list are calculated according to the following formula:
$$Z = \frac{Var - {μ}_{éta1} ( r )}{{σ}_{éta1} ( r )}$$
where functions μ_{étal}(r) and σ_{étal}(r) are obtained by smoothing of averages μ(r) and of standard deviations σ(r) previously calculated based on the normalized calibration variation values.

20. A method for analyzing the variations of mRNA concentrations of a set of genes based on m identical so-called reference cell groups (GR₁ to GRₘ) and q identical groups of so-called test cells (GT₁ to GT_{q}), the method comprising the steps of:
measuring, for each reference group, the messenger RNA concentration for each of the genes and writing the results in m reference lists (L_{ref1} to L_{ref2});
measuring, for each test group, the messenger RNA concentration for each of the genes and writing the results in q test lists (Lₜₑₛₜ₁ to Lₜₑₛₜ₂);
defining for each of the lists a rank value for each gene according to the method comprising the four steps of:
classifying the genes by increasing mRNA concentrations;
assigning a zero rank value to all genes having mRNA concentrations smaller than or equal to a threshold concentration value;
assigning a single rank value to all the other n1 genes having an mRNA concentration greater than the threshold concentration value, the rank value ranging between 1 and n1, rank R of a gene being all the higher as the mRNA concentration of said gene is high; and
normalizing the rank values over a range from 0 to w, w being a positive integer, rank r of a gene being now equal to (R*w)/n, where n is the number of studied genes,
defining a global reference list associating with each gene a single rank equal to the average of its ranks in the reference lists;
defining a global test list associating with each gene a single rank equal to the average of its ranks in the test lists;
calculating for each gene a variation value (Varₖ) equal to the difference between the gene rank for the global reference list and the gene rank for the global test list;
classifying the genes in first and second groups, according to whether the genes exhibit variation values respectively corresponding to an increase or to a decrease in their ranks between the global reference list and the global test list;
calculating for each gene of the second group a new variation value (Varₖ) equal to the difference between the gene rank for the global test list and the gene rank for the global reference list;
calculating for each gene a normalized variation value (Zₖ) according to the method comprising the two steps of:
assigning a single rank value r to each gene equal to the rank value of the reference list for genes of the first group and equal to the rank value of the test list for genes of the second group;
calculating normalized calibration variation value Zₖ of the gene according to the following formula:
$$Z = \frac{Var - μ ( r )}{σ ( r )}$$
where Var is the calibration variation of said gene, μ(r) and σ(r) respectively are the average and the standard deviation of a set of variation values corresponding to a set of genes having ranks close to rank r of said gene; and
identifying the genes exhibiting significant mRNA concentration variations from the normalized variation values.

21. The method of any of the foregoing claims, in which one or several reference, test, or calibration lists are obtained according to a method for creating an artificial data set comprising the steps of:
implementing steps h) to k) of claim 3 providing a cumulative calibration frequency distribution;
defining for each gene a normalized variation value by performing a random drawing from the cumulative calibration frequency distribution, the set of the normalized variation values thus defined having a cumulative frequency distribution identical to the calibration frequency distribution.
